SEQUENCE LISTING (8 sequences)

SEQ ID NO: 1 - DNA, 69 bp, Artificial Sequence
  Description of Artificial Sequence Synthetic probe
tcaagagtagacacaggatcagcataggcaatagatggagtcacgtaatcagatcagagcaataggtca

SEQ ID NO: 2 - DNA, 23 bp, Artificial Sequence
  Description of Artificial Sequence Synthetic probe
atggtagatggttggtatgggta

SEQ ID NO: 3 - DNA, 26 bp, Artificial Sequence
  Description of Artificial Sequence Synthetic oligonucleotide
cgtaggcaatagatggagtcactacg

SEQ ID NO: 4 - DNA, 56 bp, Artificial Sequence
  Description of Artificial Sequence Synthetic primer
aattctaatacgactcactatagggagaaggtgacctattgctctgatctgattac

SEQ ID NO: 5 - DNA, 44 bp, Artificial Sequence
  Description of Artificial Sequence Synthetic primer
taatacgactcactataggtgacctattgctctgatctgattac

SEQ ID NO: 6 - DNA, 25 bp, Artificial Sequence
  Description of Artificial Sequence Synthetic primer
tcaagagtagacacaggatcagcat

SEQ ID NO: 7 - DNA, 49 bp, Artificial Sequence
  Description of Artificial Sequence Synthetic oligonucleotide
gtgactccatctattgcctaaaaaaatacccataccaaccatctaccat

SEQ ID NO: 8 - DNA, 57 bp, Artificial Sequence
  Description of Artificial Sequence Synthetic oligonucleotide
attccctcccaaccattttctatgaaaaaaaacacatcataagggtaacagttgctg

(19) United States
(12) Patent Application Publication
(10) Pub. No.: US 2013/0137108 A1
(43) Pub. Date: May 30, 2013

(54) MAGNETIC BEAD SEPARATION APPARATUS AND METHOD

(71) Applicants: Anubhav Tripathi, Northboro, MA (US); Stephanie E. McCalla, Providence, RI (US); Jinkee Lee, Cranston, RI (US)

(72) Inventors: Anubhav Tripathi, Northboro, MA (US); Stephanie E. McCalla, Providence, RI (US); Jinkee Lee, Cranston, RI (US)

(21) Appl. No.: 13/731,338

(22) Filed: Dec. 31, 2012

Related U.S. Application Data
(63) Continuation-in-part of application No. 13/660,427, filed on Oct. 25, 2012.
(60) Provisional application No. 61/551,087, filed on Oct. 25, 2011.

(51) Int. Cl. C12Q 1/68

(52) U.S. Cl. 435/6.12; 435/293.1

(57) ABSTRACT

Disclosed herein is a diffusion-limiting reactor having a first element and a closure element, said reactor having at least two interconnected reservoirs said interconnection being by non-impinging microchannel, and at least one said reservoir and said microchannel being magnetic accessible. Further disclosed is a method of sample separation.

### CROSS-REFERENCE TO RELATED APPLICATIONS

This application claims priority to copending U.S. patent application Ser. No. 13/660,427 filed Oct. 25, 2012 and to U.S. Patent Application Ser. No. 61/551,087 filed Oct. 25, 2011.

### SEQUENCE LISTING

The instant application contains a Sequence Listing which has been submitted in ASCII format via EFS-Web and is hereby incorporated by reference in its entirety. Said ASCII copy, created on Dec. 5, 2012, is named B19729US.txt and is 2,307 bytes in size.

### FIELD OF THE INVENTION

Disclosed herein is a diffusion-limiting reactor having a first element and a closure element, said reactor having at least two interconnected reservoirs said interconnection being by non-impinging microchannel, and at least one said reservoir and said microchannel being magnetic accessible. Further disclosed is a method of sample separation.

### BACKGROUND

The target-probe or sample microbead diagnostic field has long sought a technology capable of the rapidly moving beads by other than relying on flow. An ideal system would be capable of taking less than 1 min to move thousands of beads in a microfluidic environment. Such technology would largely avoid diffusional mixing. This can be expressed as channel length (L) which will be is long so that time of diffusion=(L*L)/2D is more than 60 min. D is the diffusivity of molecule.

Available separatory chips employ reservoirs with a channel connecting said two reservoirs. The channel contains the same solution as one of the reservoirs. The reservoirs contain different solutions.

Particular reference is made to U.S. Pat. No. 7,708,881 to Yu. In Yu, the separation scheme requires multiple washes and elusion by a series of reaction chambers connected by round microfluidics channels. Yu discloses 50 um-500 um diameter channel connection with openings but does not provide any length. Their large diameter connections and short lengths permits significant molecular diffusion or mixing. Thus, the mixing solution, buffer solution and elusion solution, having mixed, ineffectively wash the beads. There is no optimal design of microtube connector between reservoir for magnetic movement in Yu. The beads bunch up and likely to clog channel. Large number of beads would lead to clogs and loss of beads in reservoir. Also, magnetic beads are pulled to the top limiting washing

Yu discloses a PMMA and Teflon device. The bonding between PMMA and Teflon will not provide perfect sealing. Both PMMA and Teflon are hydrophobic. This fosters bubble traps which are hard to exclude. Also PMMA and Teflon present surfaces which are hard to functionalize. Silicon based materials lend themselves to non specific binding of bio molecules.

Yu does not disclose employing different viscosity fluids in channels. The disclosed designs are not suitable for small electric field or gravity driven opposing flow, and offer no teaching or suggestion by which to temperature control reservoirs fluid by metal coating based methodologies. U.S. Pat. No. 7,708,881 and all publications cited herein are incorporated by reference in their entirety.

Reference is made to European Patent Application No.10800660.2 (PCT/US2010/042426) entitled DETECTION OF SHORT RNA SEQUENCES (Tripathi, et al,) published on Jun. 13, 2012 under Publication No. 2462236 disclosing an assay termed a SMART assay.

### SUMMARY OF THE INVENTION

The disclosed microfluidic sample enrichment and target detection technology provides the following attributes to the problem at hand:

(a) It is not a standard round tube based separation technique nor does it require repeated washing/rinsing steps or excessive reagents. It does not use lateral flow.

(b) The rapid travel of magnetic beads (at least about 0.5 mm/sec and preferably at least about 0.7 mm/sec) through microchannel fluids provides limited carry-over of target or sample molecules not bound to microbeads.

(c) It does not require flow of sample solution containing target molecules. The capture/binding of spores occurs in the output reservoir, so that the method uniquely processes large amount of sample for rapid separation;

(d) It separates at high efficiency without membrane or pumps; Thus, it is quite useful for resource limited settings;

(e) To reduce the carry over or diffusion of unbound molecules, a counter flow of clean high viscosity buffer from output reservoir to input reservoir is optionally created by fluid volume differential;

(f) Multiple washes are be generated by fabricating a microchannel from output reservoir to an additional output reservoir and so on (FIG. 2);

(g) The method can be fully automated using motion control hardware;

(h) The method can be integrated to a downstream microfluidic process; and,

(i) A magnetic stirrer can be used inside the reservoirs for mixing.

As to high viscosity reaction fluids or buffers, particular reference is made to viscosities of from 10× to 400× (10 times to 400 times) the viscosity of water with particular reference to the 20× to 200× range (dynamic viscosity). As to viscosity enhancers, specific reference is made to methylcellulose, hydroxypropylmethylcellulose, poly(vinlypyrrolidone) and poly(vinly alcohol). In some embodiments water is viscosity enhanced by addition of said viscosity enhancers.

Particular attention is drawn to the capacity for the disclosed fluid filled system to comprise distinct fluids at particular locations. This provides altered or reduced mixing or diffusion associated with differing fluid viscosities. In a particular embodiment it is contemplated that one reservoir have a solution of specific salinity, pH, rehology, temperature, etc as well as other components (e.g., proteins, nucleotides, epitopes, viral, ligands, or other molecules) while the microchannel leading to a next reservoir have the same or different fluid (or multiple fluids in specific sections), and the outflow reservoir have, perhaps, yet another fluid composition. And, of course, an elaborate system of multiple reservoirs and multiple microchannels, in line or branching, is also contemplated.

In a specific configuration the reactor process entails moving the magnetic beads between reservoirs by the use of an electrical field of <30 V/cm. A field of this density avoids significant non-specific binding. An optimized electric field is not strong enough to unbond target molecules but strong enough to unbond nonspecific molecules on the magnetic beads.

The invention further comprises a method of sample separation by fluid filled diffusion-limiting reactor having a first element and a closure element, wherein the reactor has at least two interconnected reservoirs. In this embodiment the interconnection is a non-impinging microchannel leading between at least two reservoirs. The microchannel is magnetic accessible. In this method of separating the samples the method comprises moving samples while the samples are affixed to magnetic beads. The method includes mixing a first solution at least incorporating a plurality of the magnetic beads under conditions wherein the sample binds with the magnetic beads in the first reservoir. The method next moves the magnetic beads using magnetic force. The magnetic beads are moved from one reservoir through said interconnecting micro channel add deposited in the next (or subsequent) reservoir. Particularly noted in this method is employing high viscosity fluid as part of the fluid fill. Note is made of viscosities of at least about 10× to about 400× the viscosity of water, and particularly from about 20× to about 200× the viscosity of water. Useful viscosity enhancers include, without limitation, methylcellulose, hydroxypropylmethylcellulose, poly(vinlypyrrolidone) and poly(vinly alcohol), alone or in combinations. Without being bound by any particular theory, faster bead movement reduces diffusion of material. It is also more efficient than fluid flow modalities to quickly conclude material separation Particular note is made of moving magnetic beads by magnetic force through said microchannel comprises moving at about at least about 0.5 mm/sec, and preferably at least about 0.7mm/sec.

### BRIEF DESCRIPTION OF THE DRAWINGS

FIG. 1 is a diagram of a two chamber magnetic bead device.

FIG. 2 is a schematic of Isolation microchip for extracting swab sample. Magnetic beads are moved from reservoir W1 (lysis buffer, 70% GuSCN) to W2 (Isopropanol and ammonium acetate) to W3 (70% ethanol) to W4 (Elution, RNase Free H20). W5 is loaded with capture beads and SMART probes. W6 is loaded with SMART reagents.

FIGS. 3A and B are reactors of microchip design for microfluidic separation and on chip amplification.

FIG. 4A. is a graph of DNA probe amplification by the present apparatus and method.

FIG. 4B. is a graph of the effect of acetate and chloride ions in the material of FIG. 4A.

FIG. 4C. is a real-time graph of fluorescence generated by and H5 probe the material of FIG. 4A.

FIGS. 5A-C are graphic presentations of system sensitivity.

FIG. 6 is graphic presentation of system sensitivity.

FIG. 7(a) and (b) are schematics of the SMART assay.

FIG. 7(c) is a diagram of three phases of a separation process.

FIG. 7(d) is a diagram of probe amplification.

FIG. 8(a) is a diagram of a chip or reactor design of the present invention and FIG. 8(b) is a photograph of the reactor.

FIG. 9(a)-(c) are graphs of probe amplification (fluorescence) data.

FIG. 10(a)-(c) are graphs of probe sensitivity (fluorescence) data.

FIG. 11 are graphs of probe sensitivity with capture of fRNA.

### DETAILED DESCRIPTION OF THE INVENTION

The present system provides rectangular or square microchannels of a taper design for efficient bead travel. The taper design also reduces the need for strong magnets. Further noted is the use of high viscosity liquids in channels. This avoids or reduces carryover of unwanted molecules. This is facilitated by the motive force being magnetic force or field and not (or only minimally) fluid flow. In the disclosed embodiments, individual reservoirs can be accurately temperature controlled. In one embodiment temperature control is by metal coating or film on a reactor glass cover and induction heating.

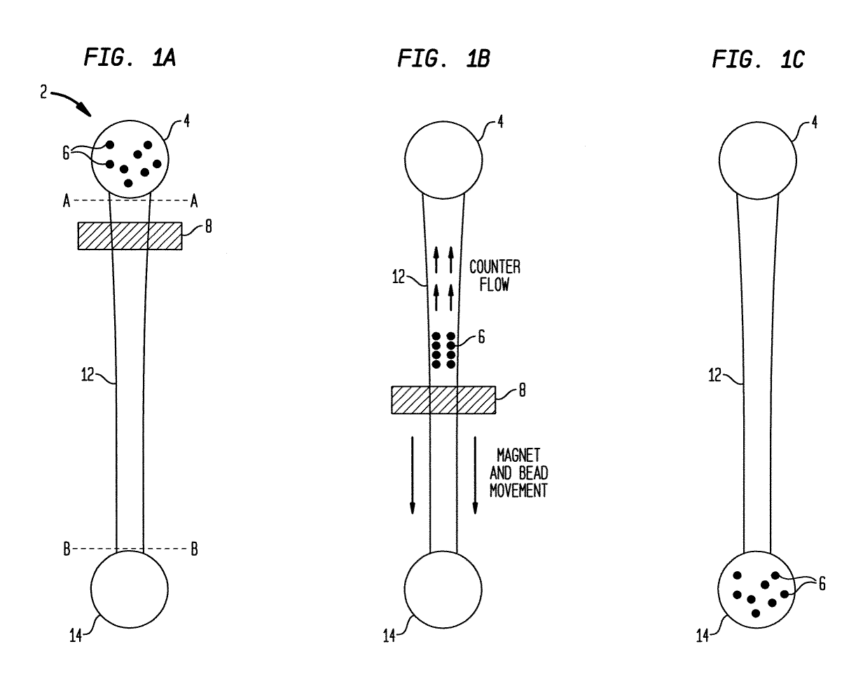

A significant aspect of this separation method is utilization of magnetic beads for bioassays, not as a static solid-support, but as a mobile platform on which multi-step microfluidic procedures are be performed. In one embodiment, magnetic beads bearing a capture antibody/nucleotide and detector antibodies/nucleotide probes are mixed in with a sample containing target molecules in the microfluidic reservoir 1 (FIG. 1). Following an incubation period, where the target molecules are bound to the magnetic bead, the whole magnetic complex (bead-antibody/nucleotide-target-detection antibody/probe complex) is moved. The magnet is moved towards the output reservoir (14), moving the beads through a microchannel containing appropriate high viscosity buffer, effectively separating the capture complex from unbound molecules. Capture complexes are finally deposited in output reservoir (14) and detected by fluorescence of the detection antibody/nucleotide. Only target-bound detection antibody/nucleotide will be present in output reservoir (14), and only if the target was present in input reservoir (4) in the sample to begin with.

This invention will be better understood with reference to the following definitions:

A. Diffusion-limiting shall mean bead migration is accomplished in least than 60 minutes, and preferably less than 30, minutes and most preferably in less than about 1 minute. Since the diffusion time t_{diff}˜l²/2D in a l=30 mm long microchannel is much longer (hours) than the total time of bead transfer t_{BT}˜1 min, the disclosed reactor and process exhibits limited or no diffusion of molecules.

B. Reactor shall mean a first element and a closure element applied to said first element so as to enclose fluid and beads used in the operation. The first element of the reactor contains at least two reservoirs and at least one channel interconnecting the reservoirs. In one embodiment, reservoirs and interconnecting channel(s) are located on the upper surface of the first element, to which the closure element may be applied. It is to be appreciated that “upper surface” references are for convenient description, Fluid and beads may be introduced into the reservoirs, and the cover element applied, while the reactor is held in inverted or any other orientation.

It is a further condition of said reactor that of said at least two reservoirs, one is an input reservoir, and said input reservoir and associated interconnecting microchannel is magnetic accessible. The microchannels of this invention are necessarily rectangular or square.

C. Magnetic accessible shall mean that a magnet is capable of moving the microbeads of this invention from said input reservoir through said microchannel to an output reservoir.

D. In the present invention the microchannels are configured in a non-impinging taper. Non-impinging shall mean microchannel tapering from a transverse width of about 4:1. to about 3:1 with particular reference to 4:1, wherein the wider end of the microchannel is at the entry point for magnetic or ferromagnetic microbeads of from about 2 to about 3 microns-3 microns (collectively, “microbeads”) and the narrower end is the exit point for said beads. Typically the entry point of the a microchannel is from a input reservoir and the exit point of a microchannel is to an output reservoir. It is understood that in multiple reservoir reactions or schemes, magnetic beads are caused to enter an output reservoir as to a first step, but be moved from there into a further microchannel, thus rendering said output reservoir an input reservoir as to a further microchannel and reservoir.

In one embodiment the microchannel tapers from about 200-50 microns over length of about 2-3 cm. The depth of channel is from about 10 to about 120 um and is substantially the same for entire channel.

Typically microchannels of this invention employing magnetic or ferromagnetic beads of from about 2 to about 3 microms-3 microns. Attention is drawn to microchannels from about 2 to about 5 mm in length with particular reference to microchannels of about 3 to about 4 mm in length.

Note is made of microchannels rectangular in cross section with a depth of about 10 to about 120 microns, with particular reference to a depth of from about 95 to about 105 microns and particularly about 100 microns.

In one reactor embodiment the first element comprises polydimethylsiloxane (PDMS) and the closure element comprises glass. Reactors have two or more depressions or hollow areas being reservoirs and microchannels connecting at least two said reservoirs.

A polydimethylsiloxane (PDMS) microchip is usefully fabricated using soft lithography. PDMS is poured over the fabricated SU-8 structure on a silicon wafer and then incubated at 75° C. for two hours. The PDMS is removed from the mold, cleaned with ethanol and oxygen plasma (PDC-32G, Harrick Plasma Ithaca, N.Y.) and bonded to a Pyrex® slip (1.4 mm thick). The microchannel is a tapered lane of 400 μm wide down to 50 μm wide, 120 μm deep and 30 mm long. The depth of the channel is measured by surface profilometer (Dektak). 1 μL of 1 μg/μL (˜65,000 beads) magnetic beads progressing through the microchannel at 0.7±0.04 mm/s (images captured at 2× magnification). Beads are guided to the beginning of the channel (left, ˜400 μm width) just beyond the initial tapering from an input reservoir. Beads then are guided to the middle of the channel (middle, ˜200 μm width). Beads are finally guided the full duration of the channel (30 mm in total) and are located at the end of the channel (right, ˜50 μm width) about to enter an output reservoir.

E. Sample or target-probe as used herein shall be broadly construed as referencing proteins, nucleotides, epitopes, viral, ligands, or other molecules which are affixed or capable of being to magnet beads for separation in the process described herein. In the process of this invention, samples are separated from other materials, which may be proteins, nucleotides, epitopes, viral, ligands, or other molecules, but distinguished as not being the desired material to bind or affix to the magnetic beads. It is to be understood that the magnetic beads may be coated with a material (e.g., straptavidin) to facilitate the binding of a sample to a bead.

FIG. 1 illustrates a device and method to separate bound and unbound probe molecules using a microchip and a magnet. In FIG. 1A, reactor (2) includes input reservoir (4) containing microbeads (6), tapered microchannel 12, moveable magnet (8) and output reservoir 14. Microchannel (12) is 200 microns wide at point A-A, and 50 microns wide at point (B-B). Capture sample is placed in fluid in input reservoir (4). Microbeads (6) prepared to bind targets are placed into input reservoir (4) binding a target molecule, if present. The reservoirs and microchannel are fluid filled.

In FIG. 1B a magnet (8) is moved or pulled across microchannel (21) toward output reservoir (14) moving by magnetic force beads from input reservoir (4) through microchannel (12) to output reservoir (14). The magnet pulls beads (6) through a large volume of buffer, effectively separating unbound probe from the capture complex. Large volume references of at least about 10-50 uL vol. of fluid as compared with microchannel volumes.

In FIG. 1C, beads are deposited in output reservoir (14) containing lesser volume fluid (<5 uL) which automatically concentrates target molecules by a factor of 10. This bead bound liquid is then used for off-chip amplification or preparing for on-chip amplification.

FIG. 1D is an end-on view at line B-B. Rector (2) is seen to have a first element (16). In this embodiment the first element is polycarbonate. Applied to the first element is a closure element (18). In this embodiment the closure element is a glass cover. Microchannel width (D) is 50 microns and depth (C) is 100 microns.

In the above embodiment, the microchannel connecting reservoirs are long. In one configuration the channel contains high viscosity fluid. In another embodiment, the channel contains a fluid that is not that of either connected reservoir. This, unlike known systems, provides a different flow environment for magnetic bead travel providing enhanced separation of sample from extraneous or unbound molecules.

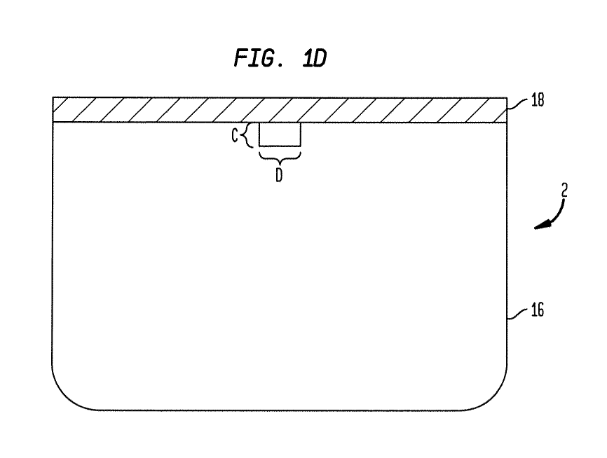

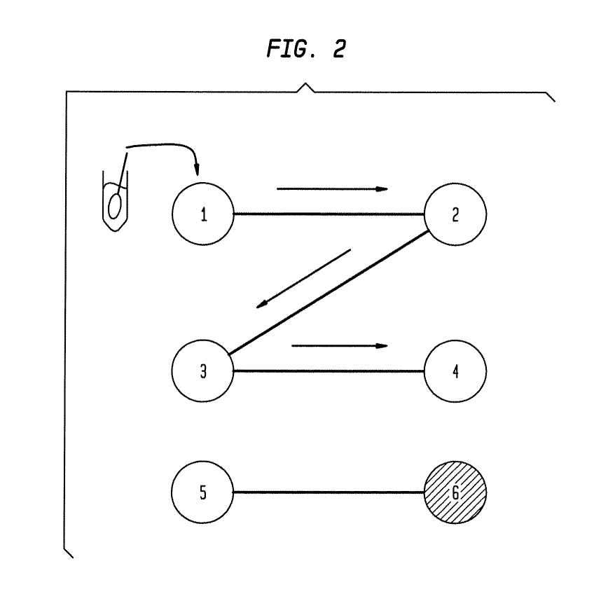

In FIG. 2 is a diagram a patient sample isolation microchip utilizing silica magnetic beads. The chip is fabricated from polymethyl methacrylate (PDMS) using soft lithography. The extraction part of chip consists of 4 wells and 4 channels which are primed with the reagents required for RNA isolation. In this embodiment, the chip does not require vacuum pressure or syringe pumps. Reagents are added to the wells with a micropipette. Capillary action fills the channels with the reagent selected. Well 1 is loaded with a specimen and lysis buffer (70% GuSCN), followed by well 2, which is primed with Isopropanol (+ammonium acetate) for a wash step. Well 3 is filled with 70% ethanol for the final step prior to elution. Well 4 is filled with nuclease free water, for elution of the RNA. Eluted sample is taken and placed at 5 for probe separation. An appropriate amount of silica magnetic beads is added to the well and a magnet is dragged along the length of the channel.

In one embodiment, a magnet (bar magnet, 2.5×1.25×0.64 cm thick) is used, directly beneath the reactor and held approximately 1 mm in front of the main bead cluster during movement. The reactor is moved relative to the magnet moving beads through the microchannel toward the output reservoir at a specific velocity. Rare earth magnets (Grade N52) were purchased from K&J Magnetics (Jamison, Pa.). Spherical super-paramagnetic streptavidin coated Dynabeads® M-280 (Ø 0 2.8 μm, 10 mg/ml) were obtained from Invitrogen (Carlsbad, Calif.).

Magnetic accessible is when the reactor is configured and adapted to move said magnetic beads between reservoirs by the use of an electrical field of about 20 to 40 V with particular reference to <30 V/cm. The non-specific binding usually contain weak interactions with bead surface. As such a small electric field breaks the interaction, likely by attracting the molecules towards positive electrode. Hence, the selected field is weak enough to unbond target molecules (while not breaking specific chemical bonds) but enough to unbond nonspecific molecules (as they are connected via weak bonds) on the magnetic beads.

The disclosed apparatus and method us useful in RNA amplification and detection using engineered amplification probes. The instant system amplifies and detects engineered ssDNA probes that hybridize to the target RNA. The amplifiable probe-target RNA complex is captured on magnetic beads using a sequence-specific capture probe and separated from unbound probe using the disclosed microfluidic technique. Hybridization sequences are not constrained as they are in target amplification reactions such as nucleic acid sequence amplification (NASBA). As disclosed herein, an ssDNA probe is amplified both off chip and in a microchip reservoir at the end of the separation microchannel using isothermal NASBA, and optimal solution conditions for ssDNA amplification Potassium and magnesium chloride are typically found in NASBA reactions. As disclosed herein, replacing the 70 mM of the 82 mM total chloride ions with acetate results in better reaction conditions in certain embodiments, particularly for low clinically relevant probe concentrations (≦100 fM). In particular embodiments of probe design and solution conditions, the initial heating step of NASBA was unnecessary, resulting in a true isothermal reaction proceeding at 41° C. for 90 minutes. The SMART assay using a synthetic model influenza DNA target sequence serves as a fundamental basis to show the efficacy of the capture and microfluidic separation system. The probes investigated here have been shown to successfully hybridize to full-length influenza A H5 vRNA, thus providing utility for addressing a clinically relevant detection problem.

Influenza is a widespread pathogen that results in approximately 36,000 annual deaths in the US. Influenza comprises variety of subtypes, which are classified by the antigenic subtype of hemagglutinin (H1-16) and neuraminidase (N1-9) proteins expressed on the viral particles. Seasonal influenza is generally caused by H1 N1, H3N2, and H1 N2 viruses, while highly pathogenic avian flu is caused by the H5N1 subtype. A swine-derived recombinant variant of H1 N1 virus has been reported in limiting a pandemic. A recurrent pandemic of influenza could result in millions of worldwide deaths. Such a catastrophic event may be avoided if rapid point-of-care diagnostics were available to health workers at the source of the infection, such that they could contain the infection before it spreads into the global community. Additionally, rapid diagnosis of influenza would prevent excessive antibiotic use, which leads to antibiotic resistant bacterial strains and is a large financial burden due to the extra cost of unnecessary treatment, hospitalization, and medication ^{9,10}.

A number of approaches have been investigated for influenza diagnostics, but each of these has associated limitations. Viral culture is specific and provides viable virus for further testing, but it takes 3-14 days for a result, limiting its use for rapid diagnostics. Immuno-specific tests, such as rapid antigen tests and immuno-fluorescence microscopy, lack sensitivity and do not provide sequence specific information for subtyping. Viral nucleic acid amplification and detection can generate subtype or strain specific information, but these tests are also complex and expensive. Polymerase Chain Reaction (PCR) requires expensive benchtop thermal cyclers and trained technicians. Nucleic Acid Sequence Based Amplification (NASBA) is faster than PCR for RNA amplification and compares favorably with PCR for HIV RNA detection in clinical samples. It is limited by secondary structure of RNA, which makes primer design and multiplexing difficult. Hybridization sites on long, clinically relevant RNA strands require stringent oligonucleotide design, with only a small fraction of sequences hybridizing efficiently to RNA. Poor hybridization of ssDNA oligonucleotides complicates primer design, making many conserved regions unsuccessful as NASBA priming sites. To combat these issues, NASBA begins with a 65° C. heating step before the addition of enzymes to disrupt full-length RNA secondary structure, and primer sites for NASBA reactions are typically 100-250 nucleotides apart to avoid products with inhibitory secondary structure.

Recently, diagnostic NASBA assays integrated with microfluidic systems have shown improvements over conventional benchtop techniques. These reactors decrease reagent use and further decrease energy consumption of the isothermal system by decreasing the reaction volume. Additionally, these small reaction volumes inherent in microfluidic chips (10 nL-2 μL) concentrate the target molecule of interest to improve primer binding and reaction kinetics. A reported study claimed the efficacy of small (10-50 nL) reaction volumes in silicon-glass reaction chamber, followed by a reactor containing 10 parallel 80 nL chambers that filled using capillary action. A particular reactor design contained 11 parallel channels with two separate chambers for each heating step separated by hydrophobic burst valves. The second chamber included dehydrated enzymes, which were shown to function in a real-time NASBA reaction upon the addition of off-chip heated reagents and primers. Although the full reaction was not run on chip, the sample was purified off-chip, and the capillary loading resulted in the loss of sample, this study shows several advantages of a microfluidic diagnostic system. The reactor was made to be simple, multiplexed, automated, shelf-stable, and consume fewer reagents and energy than its benchtop counterpart. Another reported assay integrated purification and amplification of RNA using a polydimethylsiloxane (PDMS) chamber reactor. Two reactors were contained on one chip, which would allow two RNA samples to be simultaneously purified from bacterial lysate, mixed with reaction mix, heated, mixed with enzyme, and detected real-time using sequence-specific molecular beacons. Both systems show the ability of microfluidics to create a closed, efficient, automated system that would decrease technician time, human error, and potential for contamination. These papers show the viability and advantages of incorporating a microfluidic platform with NASBA.

Disclosed herein is an amplification method, SMART, that both incorporates the advantages of microfluidics and avoids the limitations in NASBA, which include inhibitory secondary structure of the amplified RNA segment and constrained binding sites between 100 and 250 nucleotides apart. SMART achieves this by incorporating a microfluidic separation step, which allows the two binding sites to be anywhere along the RNA target. Additionally, the incorporation of an engineered ssDNA probe allows the user to choose amplifiable probe and primer sequences, which reduces secondary structure and enables the user to optimize reaction kinetics. Typically, the amplification step in SMART does not directly amplify the starting RNA.

To demonstrate this novel method, experiments were first performed off chip to verify that the amplification step created the expected nucleic acid products and to optimize solution conditions. Upon verification of an optimized amplifiable probe set and solution conditions, the optimized amplifiable probe set and solution conditions were then transferred to the microchip platform to investigate microfluidic separation and amplification. The technique achieved separation of bound probe from free probe by magnetically dragging beads through a microfluidic channel. Optimized real-time amplification and detection of the probe on-chip was also investigated.

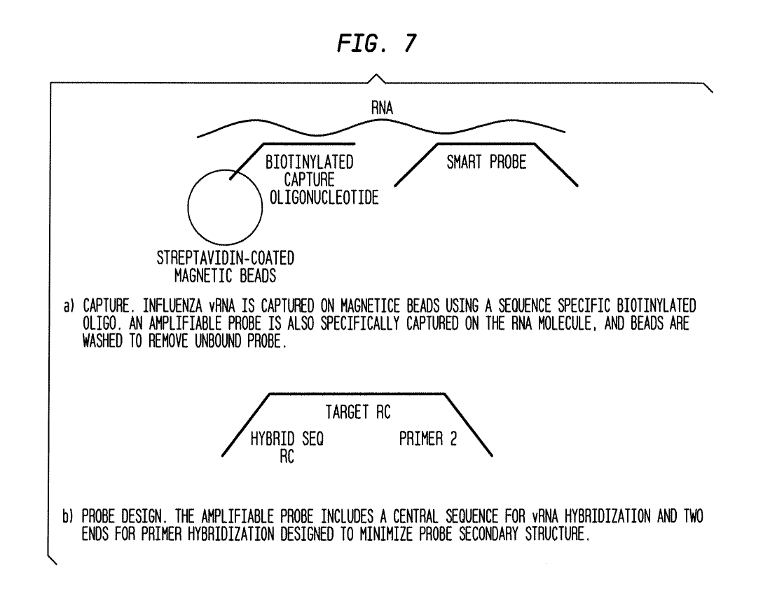

The SMART method employs two probes that bind to specific sequences within the target RNA, allowing investigators to test for multiple characteristics of a pathogen such as subtype, drug resistance, and strain. FIG. 7 shows the major components of the SMART assay. A capture step (FIG. 7a) associates bound probe molecules with a magnetic bead by hybridizing target RNA to a capture probe, consequently improving specificity due to the additional binding site needed. The sequence of the two flanking ends of the SMART probe (FIG. 7b) can be adjusted by the user in order to optimize amplification reaction kinetics. Due to the high probe amplification efficiency, separation via a microfluidic chip platform is important to minimize contamination with unbound probe molecules. The assay includes a method for separating unbound probe molecules using a PDMS chip and a magnet (FIG. 7c). Capture sample is placed in well W1, and unbound probe is separated by holding a magnet to the bottom of the microchip and slowly moving it to well W2, dragging the bead-nucleic acid complex through a large volume of buffer.

Amplification of the probe is performed via NASBA (FIG. 7d), starting with the ssDNA amplifiable probe (FIG. 7b). As with standard NASBA procedures, two primers are used in conjunction with three enzymes: AMV-RT, T7 RNA polymerase, and RNase H. We demonstrate amplification both on chip and off chip in a tube.

### Materials and Methods

TABLE 1

| | | SEQ |
|---|---|---|
| | | ID |
| Oligo | Sequence (5′→3′) | NO: |
| Amplifiable | TCAAGAGTAGACACAGGATCAGCATaggcaataga | 1 |
| Probe | tggagtcacGTAATCAGATCAGAGCAATAGGTCA | |
| Capture | /5BioTEG/ATGGTAGATGGTTGGTATGGGTA | 2 |
| Probe | | |
| Beacon | [6~FAM]CGTAGGCAATAGATGGAGTCACTAC | 3 |
| | G[BHQ1a~6FAM] | |
| Primer 1 | AATTCTAATACGACTCACTATAGGGAGAAGGTGA | 4 |
| (Optimal) | CCTATTGCTCTGATCTGATTAC | |
| Primer 1 | TAATACGACTCACTATAGGTGACCTATTGCTCTG | 5 |
| (Alternate) | ATCTGATTAC | |
| Primer 2 | TCAAGAGTAGACACAGGATCAGCAT | 6 |
| ssDNA | GTGACTCCATCTATTGCCTAAAAAAATACCCATA | 7 |
| target-H5 | CCAACCATCTACCAT | |
| ssDNA | ATTCCCTCCCAACCATTTTCTATGAAAAAAAACA | 8 |
| target-H3 | CATCATAAGGGTAACAGTTGCTG | |

Table 1: Oligonucleotides used in the SMART method, shown 5′ to 3′. Probes, primers, and molecular beacon used in the NASBA reactions target H5 vRNA, but for proof-of-concept a short ssDNA synthetic target with H5 binding sites was used. The H3 ssDNA target contains binding sites specific for H3 vRNA, which serves as a negative control for H5 ssDNA target identification. The amplifiable probe sequence is conserved in 93.6% of all sequenced human H5 strains, and the capture probe is conserved in 89.3% of all sequenced human H5 strains. The lower-case region in the amplifiable probe corresponds to a binding site in H5 vRNA, the bold regions are the primer 1 binding sites, and the underlined region corresponds to the T7 RNA polymerase promoter. The beacon contains a FAM molecule on the 5′ end and a black hole quencher molecule on the 3′ end.

Design of Capture Probes, SMART Probes, Primers, and Molecular Beacons

Custom oligonucleotides given in Table 1 were purchased from either Integrated DNA Technologies, Inc. (Coralville, Iowa) or Eurofins MWG Operon (Huntsville, Ala.). The probe portion complementary to the H5 RNA target was chosen to be a sequence that has been previously shown to bind to viral RNA. Sequences of the nucleic acid arms flanking either side of the complementary portion were chosen to minimize secondary structure. This was done by first using an online folding tool (UNAFold on The DINAMelt Server, SUNY Albany) to reject sequences with predicted excessive secondary structure. The optimal probe and primer set was then chosen by finding the probe that gave the greatest product yield as shown via gel electrophoresis after a 90 minute amplification step.

Nucleic Acid Sequence-Based Amplification Reactions

The NASBA reaction was run in 200 μL polypropylene tubes, in transparent plastic cuvettes, and in 3 mm diameter PDMS microwells. Reaction volumes varied from 5-70 μL. The base NASBA mix contained 40 mM Tris pH 8.0, 12 mM MgCl₂ or (CH₃COO)₂Mg (MgOAc), 70 mM KCl or CH₃COOK (KOAc), 5 mM DTT, 1 mM dNTP, 2 mM rNTP, 0.2 μM each primer, and 15% DMSO at the final reaction concentrations. Real-time amplification reactions also included 50 nM final concentration of molecular beacon. The base enzyme mix contained 1.6 U/μL T7 RNA polymerase, 0.325 U/μL AMV-RT, 0.005 U/μL RNase-H, and 0.105 mg/mL bovine serum albumin (BSA) at the final reaction concentrations. To test optimized solution conditions, additional BSA and Tween-20 were added where indicated. Three different thermal scenarios were used to test optimized heating conditions: heating to 65° C. for 5 min and cooling to 41° C. for 5 min before the addition of enzymes, heating to 41° C. for 2 min before the addition of enzymes, and mixing the reaction mix and enzymes at room temperature. Off-chip heating was achieved using a MyCycler thermal cycler (Biorad, Hercules, Calif.) or a water bath housed within a fluorometer (Photon Technology International, Birmingham, N.J.). In-well heating was achieved using a Thermal-Clear 0.58×2.20 in. thin film heater (Minco, Minneapolis, Minn.) affixed to the bottom of the microreactor (FIG. 2a). NASBA reactions were run at 41° C. for 90 minutes, and products were detected both in real-time by molecular beacon fluorescence and by diluting the sample 1:5 in RNase-free water and running a small RNA gel electrophoresis (Agilent Technologies, Santa Clara, Calif.).

PDMS Reactor Fabrication

Masks were created in AutoCAD (Autodesk, San Rafael, Calif.) and negative masks were printed at 20,000 dpi (CAD/Art Services, Bandon, Oreg.). SU-8 100 (MicroChem Corp., Newton, Mass.) was spin coated at a thickness of 120 μm onto 2 inch silicon wafers, prebaked at 65° C., softbaked at 95° C., and exposed to UV light in the presence of the negative mask to selectively crosslink channels. The wafer was then baked again, placed in SU-8 developer to remove uncrosslinked SU-8, and cleaned using isopropanol. The resulting master mold was used to create PDMS microfluidic reactors (FIG. 2). Sylgard 184 elastomer base was mixed 1:10 with curing agent (Dow Corning, Midland, Mich.), poured over the mask, and cured for 1 hr at 75° C. 3 mm diameter holes were punched into the PDMS reactor to form wells. The PDMS reactor and a 1 mm thick soda-lime glass slide (Corning, Corning, N.Y.) were cleaned with ethanol and treated with plasma for 1 min 40 s using a Harrick 32G plasma cleaner under 30 in Hg vacuum at high RF (Harrick Plasma, Ithaca, N.Y.). The channels were placed face down onto the glass slide, forming an irreversible bond between the plasma-treated glass and PDMS. Baking the PDMS reactor at 70° C. for at least 30 min after joining appeared to improve the glass-PDMS bond.

Capture of Target Synthetic ssDNA

The short synthetic strand of ssDNA, amplifiable probe (Eurofins MWG Operon, Huntsville, Ala.), and a biotinylated H5 capture probe (Integrated DNA Technologies, Coralville, Iowa), shown in Table 1, were mixed in a hybridization buffer composed of 20 mM Tris pH 8.0, 20 mM MgCl₂, 150 mM NaCl, 0.02% Tween-20, and 1 mg/mL BSA. A negative control sample contained an ssDNA target specific for H3 vRNA, and a bead control contained only the capture probe. The mixtures were heated to 70° C. for 2 minutes and cooled to 4° C. for 5 min before the addition of 2.8 μm diameter M-280 magnetic beads (Invitrogen, Carlsbad, Calif.) for a final volume of 20 μL. The samples were then incubated at room temperature for 35 min. Beads were collected at the bottom of the tube using cylindrical neodymium magnets (K&J Magnetics, Inc., Jamison, Pa.), and 18 μL of the supernatant was removed before transfer of the beads to the microfluidic separation platform.

Separation of Probe Complex

Separation of the bead-probe complex (FIG. 7a) from free amplifiable probe molecules (FIG. 7b) was achieved using a custom microfluidic reactor (FIG. 8). Reactor cleaning and preparation was achieved by pressurizing the well with a 1 mL syringe and first flushing the channels with nuclease decontamination solution, then flushing three times with nuclease-free water (Integrated DNA Technologies, Coralville, Iowa), and flushing two times with priming buffer (1 mg/mL BSA, 0.1% Tween-20). The microchips were stored at 4° C. for at least 14 hrs. To setup a counter flow, well 2 (W2, FIG. 2a) contained 12 μL of priming buffer, well 1 (W1, FIG. 8a) contained 2 μL priming buffer, and the chip was held at an approximately 15° tilt towards W1. Beads were placed in W1 and moved through the channel by moving a neodymium magnet along the bottom of the channel at ≦1 mm/s. When beads were <1 cm from W2, the flow was stopped by the removal of solution in both W1 and W2 and the addition of 12 μL of paraffin oil to W1. For off-chip amplification experiments, the solution in W2 was replaced with 5 μL of fresh priming solution. Beads were then pulled into W2, collected in a polypropylene tube, and W2 was rinsed with an additional 6 μL of priming buffer. The beads were then collected at the bottom of the tube and all but 2 μL of solution was removed for downstream NASBA reactions. For on-chip amplification experiments, the solution in W2 was replaced with a NASBA reaction mix that included free probe molecules and beads were pulled into W2.

In-Well Real-Time Probe Amplification

Real-time amplification of ssDNA probe was conducted in a microwell using NASBA mix without separation, such that the ssDNA probe was included in the NASBA mix and added directly to the microwell (W2, FIG. 8a). To mimic SMART reaction conditions, in-well probe amplification included beads from a bead control capture reaction and a subsequent separation step as described above. The reactor channel contained priming solution and 12 μL of paraffin oil in W1. The reactor W2 contained 5 μL NASBA mix (after the addition of 0.4 μL sample mix) and 5 μL paraffin oil to avoid sample evaporation. An adjacent reactor channel contained identical solution conditions and W1 contents, but had 5 μL of priming solution and 5 μL of paraffin oil in W2. A Thermal-Clear 0.58×2.20 in. thin film heater (Minco, Minneapolis, Minn.) was affixed to the bottom of the reactor and centered under the reactor well and the adjacent well (FIG. 8a). A type K thermocouple (Omega Engineering Inc., Stamford, Conn.) was placed in the adjacent well to accurately determine fluid temperature in the wells. An external RK-80H power supply (Matsusada Precision Inc, Kusatsu City, Shiga, Japan) provided 4-5V to the heater to achieve 41° C. in the well. The reactor was placed on a Nikon Eclipse TE300 fluorescent microscope (Nikon Instruments, Inc., Melville, N.Y.) with a blue filter set (450-490 nm excitation, 515 nm long pass emission), a 4× objective, a model 814 Photomultiplier Detection System with a gain of 700V (Photon Technology International, Birmingham, N.J.), and an integrated high speed shutter (Melles Griot, Albuquerque, N.Mex.) set to open for 0.2s. A custom in-house LabVIEW (National Instruments Corporation, Austin, Tex.) data acquisition program collected PMT output at a rate of 12-15 Hz.

### Results and Discussion

Off-Chip Probe Amplification and Optimization

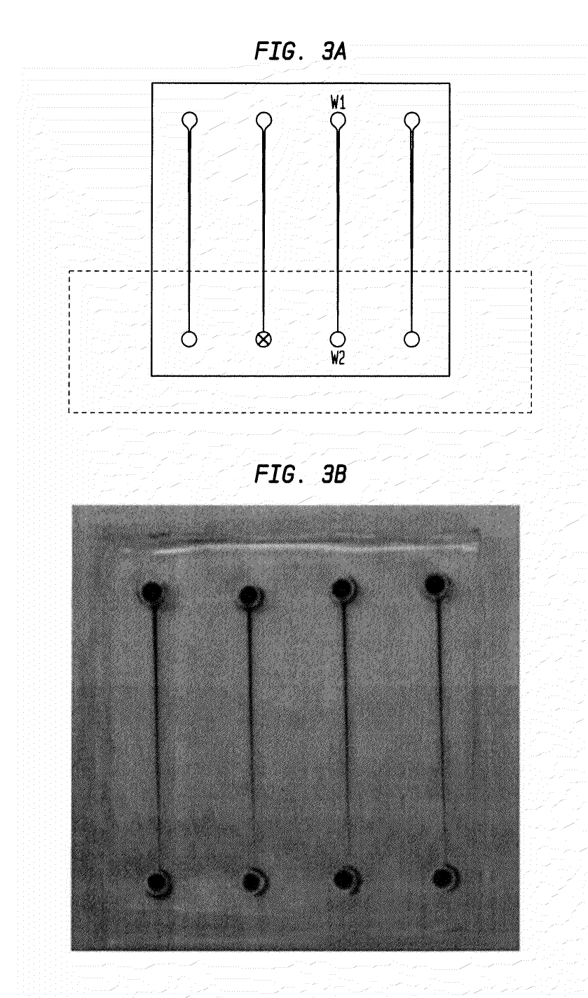

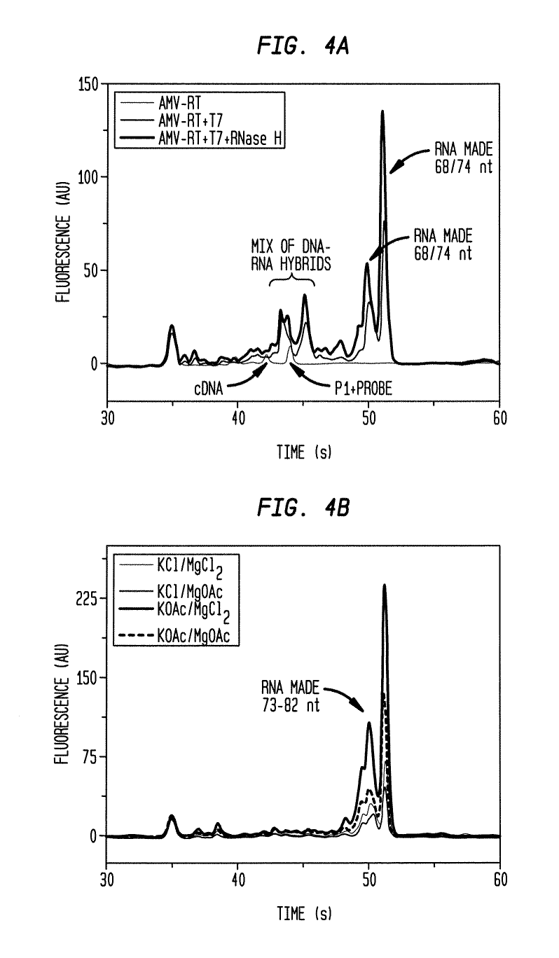

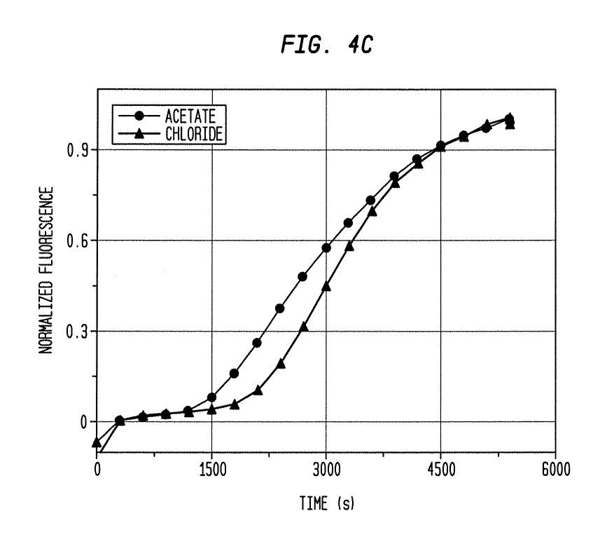

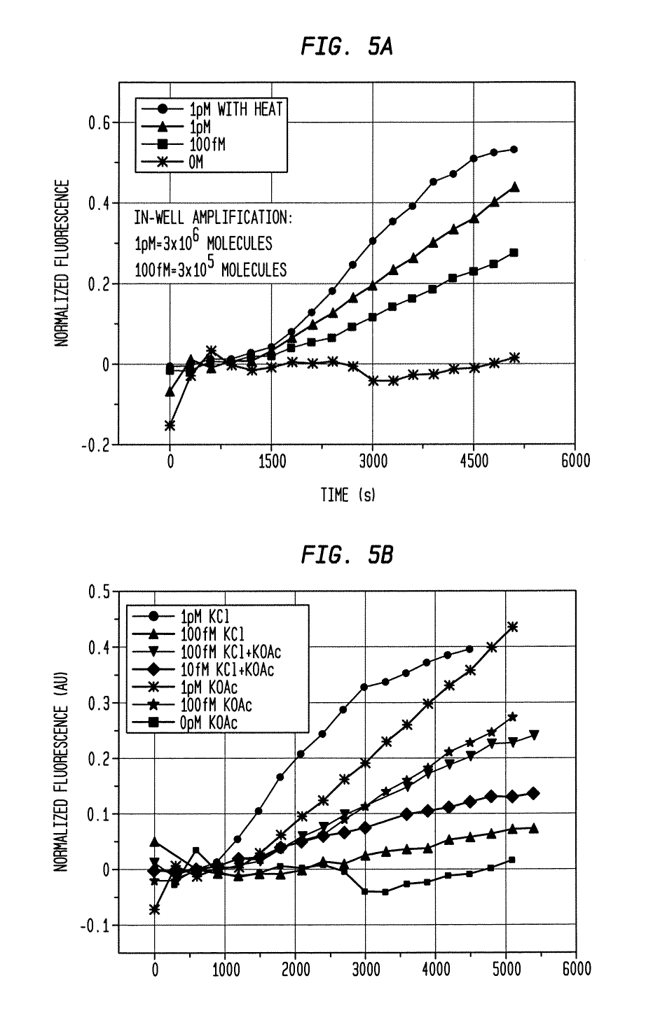

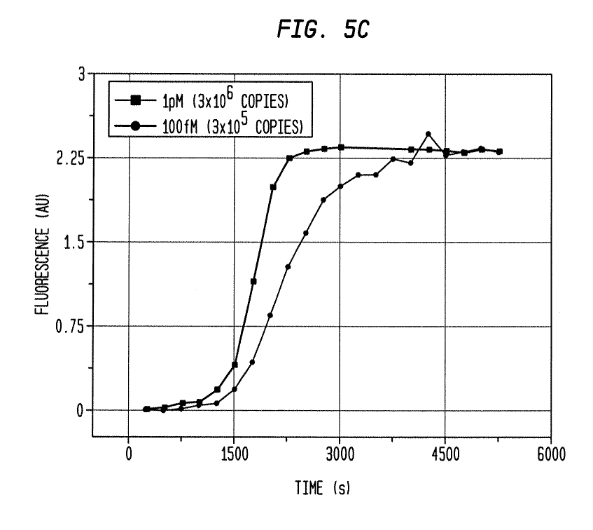

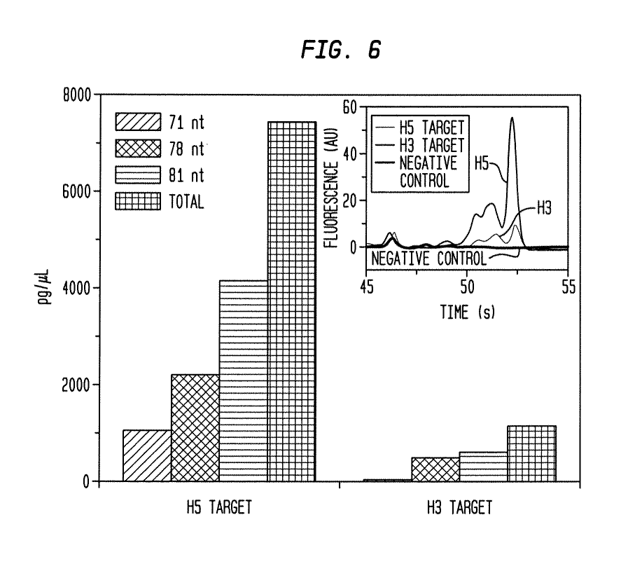

We first validated that the amplification reaction worked as designed, yielding expected products. To investigate this, combinations of starting material, including amplifiable probe, primers (primer 1 alternate and primer 2, as seen in Table 1) and dsDNA, were first characterized by small RNA gel electrophoresis to find their expected peaks and bands on an electropherogram plot. In addition, the dsDNA was transcribed in NASBA conditions with T7 RNA polymerase to find the expected band for the RNA product. Given this information, a series of tests varying the presence of enzymes with amplifiable probe and primers in NASBA conditions were performed to verify the full NASBA amplification. Three conditions (FIG. 3a) are shown: AMV-RT only, AMV-RT and T7 RNA polymerase, and all 3 enzymes. All conditions were run in KCl NASBA buffer with a 65° C./41° C. heating step before amplification. Analysis was performed by gel electrophoresis. FIG. 3a summarizes the outcomes of the experiment, confirming that the amplification reaction is occurring as expected. One important note is that the product labeled “RNA made” contains several peaks of varying length. These peaks are all confirmed to be products as they appear when the corresponding cDNA is transcribed in NASBA conditions with only T7 RNA polymerase present. We believe that these alternate peaks occur as the polymerase prematurely aborts transcription. This, however, does not affect the detection methods as the beacon targets the middle of the transcribed RNA, and thus only needs roughly the first 45 to 50 nt to successfully detect the product. No other nucleic acid species appear in the range of the RNA transcribed. Before performing the remaining experiments, the primers themselves were modified to yield the maximum product RNA. The resulting primer set was comprised of primer 1 (optimal) and primer 2, as shown in Table 1. These optimal primers were used for the remaining experiments.

A study to inspect the effect of replacing chloride ions with acetate ions was also performed to optimize NASBA for our application. To achieve this, an experiment was carried out by running 4 amplification conditions and adjusting the base NASBA mix: 1) a positive control; 2) replacing KCl with KOAc; 3) replacing MgCl₂ with MgOAc; and 4) replacing both KCl and MgCl₂ with acetate complements. Samples were subjected to 65° C./41° C. heating steps before amplification, and they were analyzed via gel electrophoresis. The data (FIG. 9b) show that replacing all of the chloride salt with acetate salt increased product yield but that a combination of 70 mM acetate and 12 mM chloride salts yields the optimum balance for these 4 conditions.

Since gel electrophoresis only allows for endpoint quantification, the previous result was investigated further to ensure that this leads to an improvement in real time detection. A positive control using KCl and an experimental sample replacing KCl with KOAc were prepared. After initial 65° C. followed by 41° C. heating steps were performed in a thermal cycler, each sample was run in a fluorometer for a 90 minute amplification reaction. The data show that the acetate sample has a faster initial rising time than the chloride sample even though the chloride sample has a faster exponential phase (FIG. 9c). The data supports using KOAc in place of KCl since it yields faster results during real-time amplification.

On-Chip Probe Amplification and Optimization

A microfluidic assay for the separation and amplification of bound probe molecules aims to simplify and streamline influenza detection, as well as save energy and reagents. The removal of the initial 65° C. heating step shortens the overall assay time, decreases the energy required to operate the device, and simplifies the heating implement necessary for the device. To determine the effects of removing this step, optimal H5 probe sequences were amplified under varied heating conditions. A full heat cycle before adding the mix to the well (65° C. for 5 min and 41° C. for 5 min before the addition of enzyme) was compared to adding the full NASBA mix to the well at room temperature and heating to 41° C. on chip for 1 pM of probe. A real-time in-well reaction that was exposed to a full heating cycle showed significantly faster exponential kinetics than the reaction that was brought to 41° C. from room temperature, although the time to positive did not appear to vary significantly between the two samples (FIG. 10a).

The addition of KCl was previously shown to increase the exponential phase of the NASBA reaction (FIG. 9c), so reincorporating KCl into the reaction mixture was also studied (FIG. 10b). KCl aided amplification without a heating step at 1 pM (3×10⁶ copies), but drastically reduced amplification without a heating step at 100 fM (3×10⁶ copies). The reincorporation of half of the original KCl concentration (35 mM KCl, 35mM KOAc) did not appear to affect real-time amplification significantly at these levels, and a linear amplification could again clearly be seen for both 1 pM and 100 fM free amplifiable probe. Increasing concentrations of either chloride or acetate ions lead to decreased transcription rates by T7 RNA polymerase, but the polymerase is more sensitive to chloride ions than acetate ions. Since the amplification reaction is cyclical, a decrease in efficiency at any given step is more pronounced at lower concentrations, and thus we believe this could explain the benefit of using KOAc in place of KCl at lower concentrations of initial probe. Without being bound by any particular theory, it is believed that it may also imply that the change of anion increased the combined efficiency of AMV-RT and RNase H in the steps of importance to the cyclic portion of amplification. In addition, it is possible that non-specific primer-dimers occur in the disclosed system, particularly given the low operational temperature and long priming sequences required. Without being bound by any particular theory, it is believed that a low, clinically relevant probe concentration with delayed amplification may be inhibited by excessive primer-dimer formation. This is particularly problematic when adding enzyme mix at room temperature. Although amplification can proceed at low probe concentrations if enzymes are added before heating the NASBA mix to 41° C., it may be advantageous to heat the sample to the reaction temperature before the addition of enzymes. These data imply that optimized solution conditions for in-well amplification of low concentration probe molecules include replacing KOAc with KCl.

In-well kinetics may also differ from benchtop reactions due to the contact of the reaction to PDMS and paraffin oil. PDMS is the main component of the device shown in FIG. 8, and paraffin oil is present to prevent sample evaporation and contamination in the reactor. Proteins have been shown to adsorb to PDMS and at oil/water interfaces, which could sequester enzyme in our system. To determine if enzyme adsorption adversely affects in-well reaction kinetics, NASBA reactions were performed using an additional 1 mg/mL BSA and 0.02% Tween-20 in the reaction mix. To prevent primer-dimer pairs, the reaction mix was heated to 41° C. before the addition of enzymes. Reaction kinetics were aided by the addition of BSA and Tween (FIG. 10c), likely due s to BSA adsorption to the hydrophobic regions of the PDMS and oil/water interface.

Capture and Amplification of Probe: SMART Assay

After determining optimized reaction conditions for in-well probe amplification, we tested the ability of the system to operate from the capture step to the amplification/detection step. 1 pM of synthetic H5 ssDNA was captured with 2 pM each of amplifiable probe and capture probe. A negative control reaction contained synthetic H3 ssDNA, and a bead control contained only capture probe (Table 1). After microfluidic separation from free probe molecules, the beads were removed from W2 (FIG. 8a), collected, and all but 2 uL of fluid was removed from the sample. The beads were transferred to a downstream 15 μL NASBA reaction; the bead control received 1 pM total of amplifiable probe to ensure that the beads and the solution conditions from the microfluidic separation did not inhibit the downstream reaction. A positive control (1 pM amplifiable probe) and negative control (0 pM amplifiable probe) were also run to ensure the reaction chemistry was robust. The samples were heated to 65° C. and cooled to 41° C. before the addition of enzymes, and the reaction was run for 90 min. The reactions were quantified on a small RNA gel electrophoresis chip. The bead control was not found to differ significantly from the positive control. A representative example of the H5 probe amplification after capture is shown in FIG. 11, with the electropherogram shown inset. The sample containing the target molecule (H5) contained 7430 pg/μL total product RNA, whereas the sample containing a false target (H3) contained 1155 pg/μL total RNA. The negative control contained no visible product RNA. Although the small peak in the H3 amplification sample indicates non-specific amplifiable probe adsorption, this small peak corresponds to a late amplification rise in the exponential NASBA kinetics, with a small rise at the end of 90 min. Thus, this system should be robust at determining the presence of target molecules, particularly when the sample is run for under 90 minutes. The non-specific adsorption may be further decreased by lengthening the separation/washing step by extending the length of the channel or manipulating the solution conditions during capture or separation.

This work demonstrates a method for amplification and detection of RNA targets by using amplifiable probes, which are engineered to optimize reaction kinetics. The reaction products, kinetics, and chemical conditions were studied utilizing both off-chip and on-chip amplification and detection systems. An optimized amplification probe was chosen, and optimized ion concentrations were determined by replacing potassium chloride with potassium acetate. Furthermore, we showed that given these optimized conditions, the initial 65° C. step can be removed, making the amplification fully isothermal, thus providing significant advantages in field and other point of care setting. The hybridization of both a capture and amplifiable probe increases specificity of the assay, and the use of magnetic beads allows for separation of the target-bead complex from unbound amplifiable probe. A simple microfluidic reactor to facilitate the separation step was also presented. Amplification in well was shown to have rapid detection at 1 pM and 100 fM with reduced reagent use, requiring only 5 μL of reaction volume. Additionally, the small reaction volume concentrates the target and probe molecules, which may decrease the time to positive. Capture, on-chip separation, and off-chip amplification was also demonstrated. Although evidence existed for non-specific binding of amplification probe, there was a distinct decrease in signal with endpoint quantification, which corresponds to a large difference in time to positive in real-time quantification for on-chip amplification. This platform is clinically relevant, as the probes presented were based upon sequences that were shown to hybridize to full-length influenza A H5 vRNA. Additionally, due to the flexibility of the amplifiable probe sequence, this method is useful for detection of any number of RNA targets, and thus this work serves as a fundamental basis for a novel, general method for RNA detection.

Note is made of the following publications incorporated here by reference in their entirety:
1. Thompson, W. W., M. R. Moore, E. Weintraub, P. Y. Cheng, X. P. Jin, C. B. Bridges, J. S. Bresee, and D. K. Shay, Estimating Influenza-Associated Deaths in the United States. American Journal of Public Health, 2009. 99: p. S225-S230.
2. Fouchier, R. A., V. Munster, A. Wallensten, T. M. Bestebroer, S. Herfst, D. Smith, G. F. Rimmelzwaan, B. Olsen, and A. D. Osterhaus, Characterization of a novel influenza A virus hemagglutinin subtype (H16) obtained from black-headed gulls. J Virol, 2005. 79(5): p. 2814-22.
3. Fiore, A. E., D. K. Shay, K. Broder, J. K. Iskander, T. M. Uyeki, G. Mootrey, J. S. Bresee, and N. S. Cox, Prevention and control of influenza: recommendations of the Advisory Committee on Immunization Practices (ACIP), 2008. MMWR Recomm Rep, 2008. 57(RR-7): p. 1-60.
4. Beigel, J. H., J. Farrar, A. M. Han, F. G. Hayden, R. Hyer, M. D. de Jong, S. Lochindarat, T. K. Nguyen, T. H. Nguyen, T. H. Tran, A. Nicoll, S. Touch, and K. Y. Yuen, Avian influenza A (H5N1) infection in humans. N Engl J Med, 2005. 353(13): p. 1374-85.
5. Bautista, E., Medical Progress: Clinical Aspects of Pandemic 2009 Influenza A (H1N1) Virus Infection (vol 362, pg 1708, 2010). New England Journal of Medicine, 2010. 362(21): p. 2039-2039.
6. Mermel, L. A., Pandemic avian influenza. Lancet Infect Dis, 2005. 5(11): p. 666-7.
7. Fauci, A. S., Pandemic influenza threat and preparedness. Emerg Infect Dis, 2006. 12(1): p. 73-7.
8. Germann, T. C., K. Kadau, I. M. Longini, Jr., and C. A. Macken, Mitigation strategies for pandemic influenza in the United States. Proc Natl Acad Sci USA, 2006. 103(15): p. 5935-40.
9. Bonner, A. B., K. W. Monroe, L. I. Talley, A. E. Klasner, and D. W. Kimberlin, Impact of the rapid diagnosis of influenza on physician decision-making and patient management in the pediatric emergency department: Results of a randomized, prospective, controlled trial. Pediatrics, 2003. 112(2): p. 363-367.
10. Woo, P. C. Y., S. S. Chiu, W. H. Seto, and M. Peiris, Cost-effectiveness of rapid diagnosis of viral respiratory tract infections in pediatric patients. J Clin Microbiol, 1997. 35(6): p. 1579-1581.
11. Dunn, J. J., et al., Comparison of the Denka-Seiken INFLU A.B.-Quick and BD Directigen Flu A+B kits with direct fluorescent antibody staining and shell vial culture methods for rapid detection of influenza viruses. Journal of Clinical Microbiology, 2003. 41(5): p. 2180-2183.
12. Petric, M., L. Comanor, and C. A. Petti, Role of the Laboratory in Diagnosis of

Influenza during Seasonal Epidemics and Potential Pandemics. The Journal of Infectious Diseases, 2006. 194: p. S98-110.
13. Gavin, P. J. a. R. B. T., Jr., Review of Rapid Diagnostic Tests for Influenza. Clinical and Applied Immunology Reviews, 2003. 4: p. 151-172.
14. Dyer, J. R., B. L. Gilliam, J. J. Eron, L. Grosso, M. S. Cohen, and S. A. Fiscus, Quantitation of human immunodeficiency virus type 1 RNA in cell free seminal plasma: comparison of NASBA™ with Amplicor™ reverse transcription-PCR amplification and correlation with quantitative culture. J Virol Methods, 1996. 60(2): p. 161-170.
15. Vandamme, A. M., S. Van Dooren, W. Kok, P. Goubau, K. Fransen, T. Kievits, J. C. Schmit, E. De Clercq, and J. Desmyter, Detection of HIV-1 RNA in plasma and serum samples using the NASBA amplification system compared to RNA-PCR. J Virol Methods, 1995. 52(1-2): p. 121-132.
16. Luebke, K. J., R. P. Balog, and H. R. Garner, Prioritized selection of oligodeoxyribonucleotide probes for efficient hybridization to RNA transcripts. Nucleic Acids Res, 2003. 31(2): p. 750-8.
17. Guatelli, J. C., K. M. Whitfield, D. Y. Kwoh, K. J. Barringer, D. D. Richman, and T. R. Gingeras, Isothermal, Invitro Amplification of Nucleic-Acids by a Multienzyme Reaction Modeled after Retroviral Replication. Proceedings of the National Academy of Sciences of the United States of America, 1990. 87(5): p. 1874-1878.
18. Deiman, B., P. van Aarle, and P. Sillekens, Characteristics and Applications of Nucleic Acid Sequence-Based Amplification (NASBA). Molecular Biotechnology, 2002. 20: p. 163-179.
19. Gulliksen, A., L. Solli, F. Karlsen, H. Rogne, E. Hovig, T. Nordstrom, and R. Sirevag, Real-time nucleic acid sequence-based amplification in nanoliter volumes. Anal Chem, 2004. 76(1): p. 9-14.
20. Gulliksen, A., L. A. Solli, K. S. Drese, O. Sorensen, F. Karlsen, H. Rogne, E. Hovig, and R. Sirevag, Parallel nanoliter detection of cancer markers using polymer microchips. Lab Chip, 2005. 5(4): p. 416-420.
21. Furuberg, L., M. Mielnik, A. Gulliksen, L. Solli, I. R. Johansen, J. Voitel, T. Baier, L. Riegger, and F. Karlsen, RNA amplification chip with parallel microchannels and droplet positioning using capillary valves. Microsystem Technologies, 2008. 14(4): p. 673-681.
22. Dimov, I. K., J. L. Garcia-Cordero, J. O'Grady, C. R. Poulsen, C. Viguier, L. Kent, P. Daly, B. Lincoln, M. Maher, and R. O'Kennedy, Integrated microfluidic tmRNA purification and real-time NASBA device for molecular diagnostics. Lab on a Chip, 2008. 8(12): p. 2071-2078.
23. Kerby, M., et al., Direct Sequence Detection of Structured H5 Influenza Viral RNA. The Journal of Molecular Diagnostics, 2008. 10(3): p. 225-235.
24. Kern, J. A. and R. H. Davis, Application of solution equilibrium analysis to in vitro RNA transcription. Biotechnology Progress, 1997. 13(6): p. 747-756.
25. Prakash, A. R., M. Amrein, and K. V. I. S. Kaler, Characteristics and impact of Taq enzyme adsorption on surfaces in microfluidic devices. Microfluid Nanofluid, 2008. 4: p. 295-305.
26. Beverung, C. J., C. J. Radke, and H. W. Blanch, Protein adsorption at the oil/water interface: characterization of adsorption kinetics by dynamic interfacial tension measurements. Biophys Chem, 1999. 81(1): p. 59-80.
27. White, Frank, Viscous Fluid Flow, (Tata McGraw Hill (2011)).
28. Z M Saiyed et al, “Application of magnetic techniques in the field of drug discovery and biomedicine,” BioMagnetic Research and Technology 2003, 1:2

1. A diffusion-limiting reactor having a first element and a closure element, said reactor having
at least two interconnected reservoirs
said interconnection being by non-impinging microchannel, and at least one said reservoir and said microchannel is magnetic accessible.

2. The reactor of claim 1 wherein the first element comprises polydimethylsiloxane and the second element comprises glass.

3. The reactor of claim 1, wherein said microchannel is tapered from about 200 microns in width and about 100 microns in depth to about 50 microns in width and about 100 microns in depth.

4. The reactor of claim 1 further comprising microbeads located in at least one reservoir or at least one microchannel.

5. A method of sample separation by fluid filled diffusion-limiting reactor having a first element and a closure element, said reactor having
at least two interconnected reservoirs
said interconnection being by non-impinging microchannel, and at least one said reservoir and said microchannel is magnetic accessible;
said method of separating said samples comprising moving samples as affixed to magnetic beads in said reactor by the steps of mixing a first solution at least incorporating a plurality of said magnetic beads a sample extraction under conditions wherein a sample binds with the magnetic beads in a first reservoir;
moving by magnetic force the magnetic beads from said first reservoir through said interconnecting micro channel; and,
depositing said beads in said second reservoir.

6. The method of claim 5 wherein said fluid comprises at least in part, a high viscosity fluid.

7. The method of claim 6 wherein said high viscosity fluid is at least about 10× to about 400× the viscosity of water.

8. The method of claim 6 wherein said high viscosity fluid comprises a member of the group comprising methylcellulose, hydroxypropylmethylcellulose, poly(vinlypyrrolidone) or poly(vinly alcohol).

9. The method of claim 5 wherein said moving magnetic beads by magnetic force through said microchannel comprises moving at about at least about 0.5 mm/sec.
